(19) United States
(12) Patent Application Publication
(10) Pub. No.: US 2025/0224406 A1
(43) Pub. Date: Jul. 10, 2025

(54) MARKERS FOR PROGRESSION OF INFLAMMATORY LIVER DISEASE

(71) Applicant: RANDOX LABORATORIES LTD, Crumlin Antrim (GB)

(72) Inventors: John Lamont, Crumlin Antrim (GB); Peter Fitzgerald, Crumlin Antrim (GB); Mark Ruddock, Crumlin Antrim (GB); Joanne Watt, Crumlin Antrim (GB)

(21) Appl. No.: 18/851,479

(22) PCT Filed: Mar. 29, 2023
(86) PCT No.: PCT/EP2023/058171

§ 371 Date: Sep. 26, 2024

(30) Foreign Application Priority Data
Mar. 29, 2022 (GB) 2204413.5

(51) Int. Cl. G01N 33/68

(52) U.S. Cl. CPC G01N 33/6863; G01N 33/6893; G01N 2333/525; G01N 2333/5412; G01N 2333/5421; G01N 2800/085

(57) ABSTRACT

The current invention provides biomarkers and combinations thereof for the diagnosis and prognosis of inflammatory liver disease. Methods include the biomarkers suppression of tumorigenicity 2 receptor (ST2), interleukin 6 (IL-6), interleukin 8 (IL-8), tumour necrosis factor alpha (TNFα) and Procollagen III N-terminal peptide (P3NP).

### BACKGROUND

Nonalcoholic fatty liver disease (NAFLD) is a condition in which excess fat accumulates in the liver of a patient with no history of alcohol abuse or other secondary causes of chronic liver disease, e.g. autoimmune, viral hepatitis (Chalasani et al., 2012; Stojsavljevic et al., 2014; Mikolasevic et al., 2016). NAFLD affects an estimated 25% of the global population. The disease affects all populations, all age groups and is the most common liver disorder in Western industrialized countries, where the major risk factors for NAFLD are common (Stojsavljevic et al., 2014). One in 3 people in the United Kingdom is estimated to be affected by NAFLD which is closely related to the increased frequency in overweight and obese individuals.

The liver helps the body maintain homeostasis by playing an essential role in lipid (fat) metabolism, i.e. fat is broken down for energy; excess glucose is converted into fat for storage. Healthy liver cells should contain little or no fat (<5%). Excessive fat accumulation in the liver causes inflammation which damages liver cells and can stimulate the progression from steatosis to nonalcoholic steatohepatitis (NASH).

The pathogenesis of NAFLD is a subject of extensive research. NAFLD is sometimes explained by a “two-hit” hypothesis. However, it should be considered as a multiple step process, with accumulation of liver fat being the first step, followed by the development of inflammation and fibrosis (Basaranoglu, Basaranoglu and Senturk, 2013). Strong epidemiological, biochemical and therapeutic evidence implicate insulin resistance (IR) as the primary pathophysiological disorder and the key mechanism leading to hepatic steatosis (Lonardo et al., 2005). Insulin actions are attenuated in IR, resulting in increased lipolysis and synthesis of free fatty acids (FFA) in the liver. Thus, accumulation of triglycerides in the liver represents the first stage or “first hit” in the pathogenesis of NAFLD. However, the progression to NASH requires the presence of additional pathophysiological abnormalities. The next step or “second hit” is the result of reactive oxygen species (ROS) that increase oxidative stress within the hepatocytes and mediate the progression from steatosis to steatohepatitis and fibrosis (collagen deposition) (Basaranoglu, Basaranoglu and Senturk, 2013). Furthermore, a “third hit” has been proposed, when oxidative stress causes progressive cell death with diminished replication of mature hepatocytes and subsequent increased progenitor cell expansion, leading to progression of liver cirrhosis and hepatocellular carcinoma (HCC) (Stojsavljevic et al., 2014).

### REFERENCES

Chalasani, N. et al. (2012) ‘The diagnosis and management of non-alcoholic fatty liver disease: practice guideline by the American Gastroenterological Association, American Association for the Study of Liver Diseases, and American College of Gastroenterology.’, Gastroenterology. United States, 142 (7), pp. 1592-1609. doi: 10.1053/j.gastro.2012.04.001
Basaranoglu, M., Basaranoglu, G. and Senturk, H. (2013) ‘From fatty liver to fibrosis: a tale of “second hit”.’, World journal of gastroenterology. United States, 19 (8), pp. 1158-1165. doi: 10.3748/wjg.v19.i8.1158.
Lonardo, A. et al. (2005) ‘Review article: hepatic steatosis and insulin resistance.’, Alimentary pharmacology & therapeutics. England, 22 Suppl 2, pp. 64-70. doi: 10.1111/j.1365-2036.2005.02600.x.
Mikolasevic, I. et al. (2016) ‘Nonalcoholic fatty liver disease-A multisystem disease?’, World Journal of Gastroenterology, 22 (43), pp. 9488-9505. doi: 10.3748/wjg.v22.i43.9488.
Stojsavljevic, S. et al. (2014) ‘Adipokines and proinflammatory cytokines, the key mediators in the pathogenesis of nonalcoholic fatty liver disease.’, World journal of gastroenterology. United States, 20 (48), pp. 18070-18091. doi: 10.3748/wjg.v20.i48.18070.

### SUMMARY OF INVENTION

A first aspect of the current invention is a method of aiding in the diagnosis of non-alcoholic steatohepatitis (NASH) or non-alcoholic fatty liver disease (NAFLD), said method comprising i) determining the level of suppression of tumorigenicity 2 receptor (ST2) in an ex vivo blood, serum or plasma sample previously obtained from a patient and ii) establishing the significance of the level of ST2 in comparison to a control. In a preferred embodiment the levels of one or more of the following biomarkers are also determined, interleukin 6 (IL-6), interleukin 8 (IL-8), tumour necrosis factor alpha (TNFα) and Procollagen III N-terminal peptide (P3NP) and their significance is also established in comparison to controls.

Optionally, each of the biomarker concentration values is inputted into a statistical methodology to produce an output value that indicates whether the patient has or is at risk of developing NASH or NAFLD.

This method can be used to differentiate between benign steatosis and NASH or NAFLD or to grade the fibrosis associated with NAFLD or NASH.

A second embodiment of the current invention is a method for monitoring the prognosis of benign steatosis to NAFLD or NASH, said method comprising i) determining the level of suppression of TNFα and ST2 in an ex vivo blood, serum or plasma sample previously obtained from a patient and ii) establishing the significance of the levels in comparison to controls. Optionally this method can further comprise determining the level of one or more of IL-6, IL-8 or P3NP.

A further embodiment of the current invention is a method for monitoring the prognosis of benign steatosis, said method comprising i) determining the level of TNFα, and optionally one or both of IL-6 and IL-8, in an ex vivo blood, serum or plasma sample previously obtained from a patient and ii) establishing the significance of the levels in comparison to controls.

### DESCRIPTION OF THE DRAWINGS

FIG. 1 (A-E) Shows plots and significance values for the five biomarkers of the invention, IL-6, IL-8, TNFα, P3NP and ST2.

FIG. 2A decision tree using the biomarker results for ST2, IL-6 and TNFα which was developed using the Rattle package of the R statistical software. This was applied to both a training and test set of the biomarker data and had an AUC value for the test set of 0.727 for distinguishing control, benign steatosis and NAFLD/NASH. Units for the ST2 cut-offs used in the decision tree were ng/ml, while the units for IL-6 and TNFα were pg/ml.

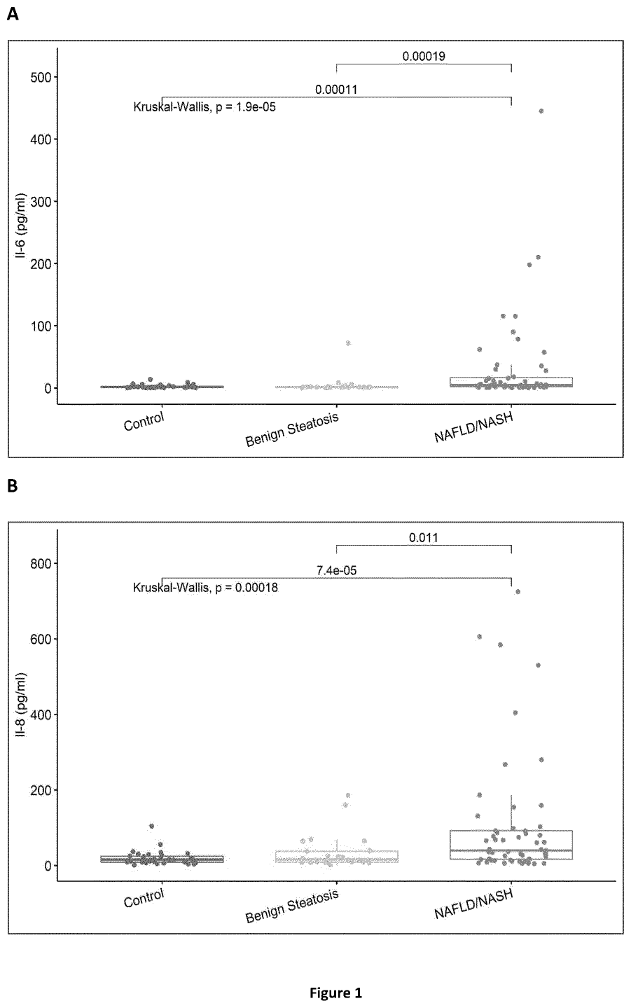

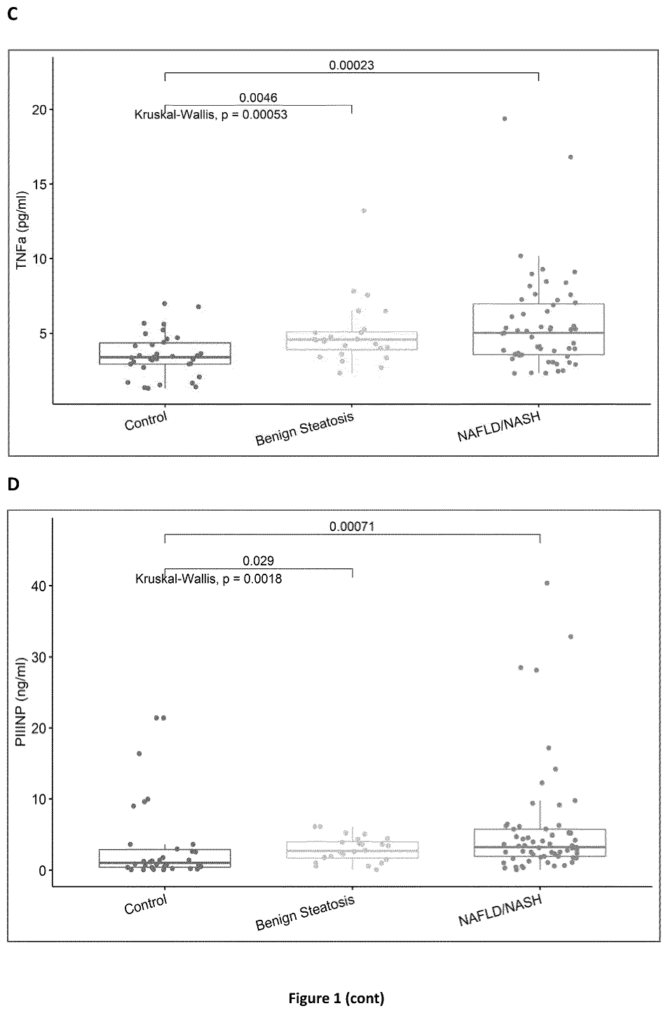

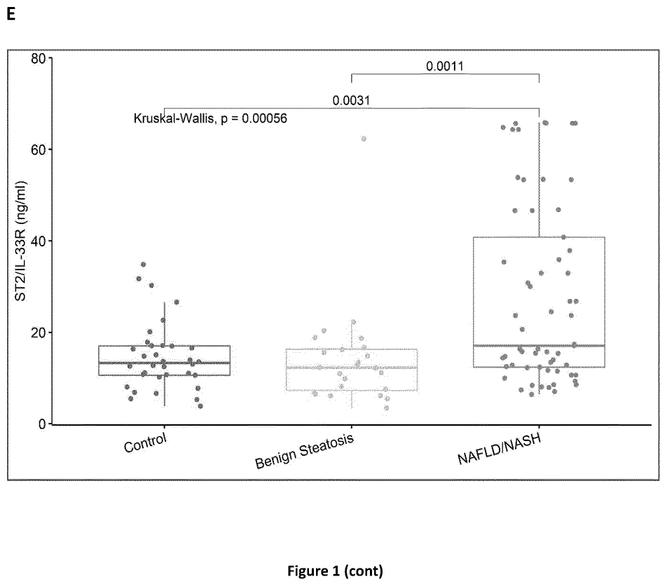

### DETAILED DESCRIPTION OF THE INVENTION

The present invention provides a method of aiding the diagnosis of NASH or NAFLD in a patient, said method comprising determining the level of ST2 and one or more additional biomarkers selected from the list consisting of IL-6, IL-8, TNFα and P3NP in an ex vivo sample previously obtained from the patient; and establishing the significance of the concentration of the biomarkers. Any two, three or four marker combinations of these biomarkers (wherein one of the biomarkers is ST2) or the combination of all five biomarkers may be useful in aiding the diagnosis of NASH or NAFLD. In a preferred embodiment of the current invention one of the additional biomarkers is TNFα. A further preferred combination of markers for the diagnosis of NASH or NAFLD includes TNFα and IL-6.

The term “biomarker”, in the context of the current invention, refers to a molecule present in a biological sample of a patient, the levels of which may be indicative of NASH or NAFLD. Such molecules may include peptides/proteins or nucleic acids and derivatives thereof. As used herein the term “determining” means quantitatively analysing for the amount of a substance present, in this case the biomarkers in a patient sample.

The term “ST2” as used herein refers suppression of tumourigenicity 2 receptor (UniProt Q01638) also known as Interleukin-1 receptor-like 1 (IL1RL1) and Interleukin 33 receptor (IL-33R). Even more specifically it refers to isoform A of ST2 (Q-01638-1) also known as ST2L. The term “IL-6” as used herein refers to interleukin 6 (UniProt P05231)

The term “IL-8” as used herein refers to interleukin 8 (UniProt P10145)

The term “TNFα” as used herein refers to tumour necrosis factor alpha (UniProt P01375).

The term “P3NP” as used herein refers to Procollagen III N-terminal peptide (The N-terminal propeptide of collagen alpha-1 [iii] chain, UniProt P02461, also referred to as PIIINP).

In the context of the present invention, a “control” or “control value” is understood to mean the level of a biomarker typically found in patients who do not have NASH or NAFLD. In one embodiment of the current invention control groups may include individuals with benign steatosis. The control level of a biomarker may be determined by analysis of a sample isolated from a person who does not have NASH or NAFLD or may be the level of the biomarker understood by the skilled person to be typical for such a person. The control value of a biomarker may be determined by methods known in the art and normal values for a biomarker may be referenced from the literature from the manufacturer of an assay used to determine the biomarker level. The control can be established as a calibration, alternatively, a calibration curve can be generated using analyte preparations at multiple concentrations. The assay signal output generated from a sample can be applied to the calibration curve to enable quantification of the analyte level of said sample.

The “level” of a biomarker refers to the amount, expression level or concentration of the biomarker within the sample. This level can be a relative level in comparison to another biomarker or a previous sample. Biomarker levels may be expressed as ratios, for example ratios between patient levels and control levels for the same biomarker or between levels of different biomarkers within the patient sample.

As used herein, the term “a sample” includes biological samples obtained from a patient or subject, which may comprise blood, plasma, serum, or urine. The methods of the invention described herein are carried out ex vivo. For the avoidance of doubt, the term “ex vivo” has its usual meaning in the art, referring to methods that are carried out in or on a sample obtained from a subject in an artificial environment outside the body of the subject from whom the sample has previously been obtained. The sample may be any sample obtained from the subject from which the biomarkers of the current invention can be determined. Preferred samples include blood samples, serum samples and plasma samples. Most preferably the sample is a serum sample.

The terms “patient” and “subject” are used interchangeably herein and refer to any animal (e.g., mammal), including, but not limited to, humans, non-human primates, canines, felines, rodents, and the like. Preferably patients are those suspected of having or being at risk of developing NASH or NAFLD.

The term “aiding in the diagnosis of” as used herein can refer to the use of the biomarker combinations described herein to differentiate between controls and benign steatosis, benign steatosis and NAFLD/NASH or between a control group which includes benign steatosis and NAFLD/NASH. A combination of ST2 and P3NP may be used to diagnosis the early stages of fibrosis associated with NAFLD/NASH or to grade said fibrosis.

In an additional embodiment of the current invention there is provided a method for monitoring the prognosis of benign steatosis, said method comprising i) determining the level of suppression of TNFα and ST2 in an ex vivo blood, serum or plasma sample previously obtained from a patient and ii) establishing the significance of the levels in comparison to controls. Said method can further comprise determining the levels of one or more of IL-6, IL-8 or P3NP. Such a method could be applied at different time intervals to monitor the progression, or lack thereof, from benign steatosis to NAFLD or NASH.

A further embodiment of the current invention provides a method for monitoring the prognosis of benign steatosis, said method comprising i) determining the level of TNFα, and optionally one or both of IL-6 and IL-8, in an ex vivo blood, serum or plasma sample previously obtained from a patient and ii) establishing the significance of the levels in comparison to controls. Such a method could be applied at different time intervals to monitor the progression, or lack thereof, from benign steatosis to NAFLD or NASH.

The concentrations of the biomarkers of the invention may be determined either sequentially or simultaneously in samples previously isolated from patients. The determination of the level of biomarkers in a sample may be determined by routine methods known in the art, such as immunological methods, for example, an immunoturbidimetric assay or ELISA based assay. Preferably, the methods of the present invention use a solid-state device for determining the level of biomarkers in the sample isolated from the patient. The solid-state device comprises a substrate having an antibody that binds specifically to a biomarker immobilised upon it. Such antibodies may be immobilised at discreet areas of an activated surface of the substrate. The solid-state device may perform multi-analyte assays such that the level of one biomarker in a sample isolated from the patient may be determined simultaneously with the level of one or more further biomarkers of interest in the sample. In this embodiment, the solid-state device has a multiplicity of discrete reaction sites each bearing a desired antibody covalently bound to the substrate, and in which the surface of the substrate between the reaction sites is inert with respect to the target biomarker. The solid-state, multi-analyte device may therefore exhibit little or no non-specific binding. Wherein one or more of the biomarkers is not compatible with a multi-analyte format they can be determined simultaneously, or indeed separately, using a suitable format such as ELISA or immunoturbidimetric assay.

A device that may be used in the invention may be prepared by activating the surface of a suitable substrate and applying an array of antibodies on to discrete sites on the surface. If desired, the other active areas may be blocked. The ligands may be bound to the substrate via a linker. In particular, it is preferred that the activated surface is reacted successively with an organosilane, a bifunctional linker and the antibody. A preferred solid support material is in the form of a biochip. A biochip is typically a planar substrate that may be, for example, mineral or polymer based, but is preferably ceramic. The solid-state device used in the methods of the present invention may be manufactured according to the method disclosed in, for example, GB patent number GB2324866. Preferably, the solid-state device used in the methods of the present invention is the Biochip Array Technology system (BAT) (available from Randox Laboratories Limited, Crumlin, Northern Ireland). More preferably, the Evidence Evolution, Evidence Investigator and Multistat apparatus (also available from Randox Laboratories) may be used to determine the levels of biomarkers in the sample.

The solid-state device comprises binding molecules attached thereto, said binding molecules having affinity specific for ST2 and, separately, one or more of IL-6, IL-8, TNFα and P3NP. In one preferred embodiment the binding molecules have affinity for ST2 and, separately, TNFα. In a further preferred embodiment there are also present binding molecules for IL-6. In another preferred embodiment the binding molecules, each in discrete locations, have affinity specific for ST2, IL-6, IL-8, TNFα and P3NP.

The present invention also provides the use of a solid-state device described herein in a method for aiding in the diagnosis of NASH or NAFLD in a patient.

The terms “immunoassay”, “immuno-detection” and immunological methods” are used interchangeably herein and refer to antibody-based techniques for identifying the presence of or levels of a protein in a sample. Examples of such assays and methods are well known to those of skill in the art.

The term “binding molecule” as used herein refers to any molecule that is capable of specifically binding to a target molecule, in this case the biomarkers, such that the target molecule can be detected as a consequence of said specific binding. Binding molecules that can be used in the present invention include, for example, antibodies, aptamers, phages and oligonucleotides. In a preferred embodiment of the current invention the binding molecules are antibodies. The term “antibody”, or the plural thereof, refers to an immunoglobulin which specifically recognises an epitope on a target as determined by the binding characteristics of the immunoglobulin variable domains of the heavy and light chains (VHS and VLS), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example Fab, Fab′, and Fr fragments, linear antibodies, single chain antibodies and multi-specific antibodies comprising antibody fragments), single chain variable fragments (scFv's), multi-specific antibodies, chimeric antibodies, humanised antibodies and fusion proteins comprising the domains necessary for the recognition of a given epitope on a target. Preferably, references to antibodies in the context of the present invention refer to polyclonal or monoclonal antibodies. Antibodies may also be conjugated to various reporter moieties for a diagnostic effect, including but not limited to radionuclides, fluorophores, or dyes.

The term “binds specifically”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition. Techniques known in the art for determining the specific binding of a target by a monoclonal antibody or lack thereof include but are not limited to, FACS analysis, immunocytochemical staining, immunohistochemistry, western blotting/dot blotting, ELISA, affinity chromatography. By way of example and not limitation, specific binding, or lack thereof, may be determined by comparative analysis with a control comprising the use of an antibody which is known in the art to specifically recognise said target and/or a control comprising the absence of, or minimal, specific recognition of said target (for example wherein the control comprises the use of a non-specific antibody). Said comparative analysis may be either qualitative or quantitative. It is understood, however, that an antibody or binding moiety which demonstrates specific recognition of a given target is said to have higher specificity for said target when compared with an antibody which, for example, specifically recognises both the target and a homologous protein.

A biomarker present in a sample isolated from a patient having NASH or NAFLD may have levels which are different to that of a control. However, the levels of some biomarkers that are different compared to a control may not show a strong enough correlation with NASH or NAFLD such that they may be used to diagnose NASH or NAFLD with an acceptable accuracy. If two or more biomarkers are to be used in the diagnostic method a suitable mathematical or machine learning classification model, such as logistic regression equation, can be derived. Such models as described herein may be referred to as “statistical methodologies”. The significance of the levels of the biomarkers can be established by inputting into said model. Such a classification model may be chosen from at least one of decision trees, artificial neural networks, logistic regression, random forests, support vector machine or indeed any other method developing classification models known in the art. The output of the models used herein would correlate with the risk of a patient having or developing NASH or NAFLD. Such an output could be a numerical value, for example a number between 0 and 1, an odds ratio value, a risk ratio/relative risk value or an alphabetic output such as ‘yes’ or ‘no’ or ‘high risk’, ‘low risk’ etc. The low risk category could be benign steatosis and the high risk category could be NAFLD/NASH. The skilled person will appreciate that the model generated for a given population may need to be adjusted for application to datasets obtained from different populations or patient cohorts.

The term “sensitivity”, used in the context of a diagnostic test, describes the percentage or ratio of subjects actually positive for the condition that are deemed positive by the biomarker test, sometimes referred to as the true positive rate. The term “specificity”, used in the context of a diagnostic test, indicates the percentage or ratio of the subjects deemed negative by the biomarker test that are actually negative for the condition (true negative rate). In these studies, it is customary for the number of positive subjects to be pre-determined by the current gold standard of testing (in this case, histopathology of biopsied tumour tissue), in order that these analyses may be performed.

One convenient goal to quantify the diagnostic accuracy of a laboratory test is to express its performance by a single number. The most common global measure is the area under the curve (AUC) of the receiver-operating characteristics (ROC) plot. The area under the ROC curve is a measure of the probability that the perceived measurement will allow correct identification of a condition. By convention, this area is typically >0.5. Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0). In the context of the present invention, the two different conditions are whether a patient has NASH/NAFLD or not. In a clinical setting it would be desirable to assign NASH/NAFLD with 100% sensitivity.

This means that the majority of subjects, who will end up outside of this category, can have NASH/NAFLD ruled out and avoid an unnecessary biopsy.

Cut-off values selected herein were those which maximised sensitivity and specificity for this dataset. It is well understood in the art that biomarker normal or ‘background’ concentrations may exhibit slight variation due to, for example, age, gender, or ethnic/geographical genotypes. As a result, the cut-off value used in the methods of the invention may also vary due to optimization depending upon the target patient or population. Adjusting the cut-off will also allow the operator to increase the sensitivity at the expense of specificity and vice versa.

Models or algorithms generated from biomarkers of the current invention could stratify presenting patients into low and high-risk categories based on a biomarker risk score (BRS). It is proposed that they could be used clinically to allow clinicians to make evidence-based decisions regarding patient management, i.e., when to refer a patient to secondary care for biopsy.

Multivariate approaches and modelling to developing a risk prediction model offer an advantage in accuracy compared to that of a single marker. Combining proteomic, genomic, and clinical measurements provide evidence-based decision making for the clinician. Incorporating clinical risk with results from the biomarkers would allow clinicians to make evidence-based decisions and assist with patient management. For example, the models could be improved further with the collection of clinical factors such as age, family history, BMI levels, and other clinical factors. For example, patients who are positive for both a biomarker risk score (as determined by any of the biomarker combinations presented herein) and a clinical risk score would be placed into a higher risk category than patients who are only positive for one of the two risk scores. Therefore, a further embodiment of the current invention is the combination of a BRS and a CRS to categorise patients into risk groups for development or progression of NAFLD or NASH.

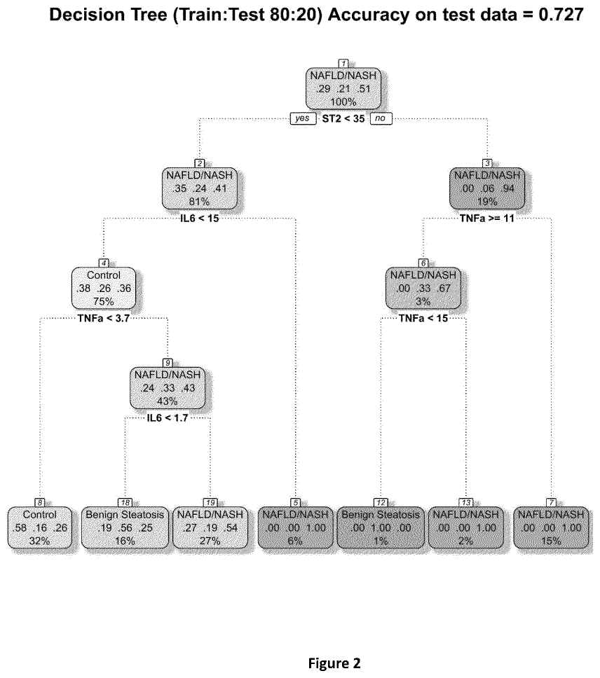

In one embodiment of the current invention a decision tree can be created using the biomarker results. FIG. 2 shows an example of such a decision tree using the biomarker results for ST2, IL-6 and TNFα which was developed using the Rattle package of the R statistical software. The preferred cut-off for ST2 is 35 ng/ml, for patients with ST2 values greater than 35 ng/ml their TNFα values are then subjected to cut-offs of 11 μg/ml and then if greater than 11 μg/ml, a further cut-off of 15 μg/ml to determine their likelihood of suffering with NAFLD/NASH or benign steatosis. For patients with ST2 values less than 35 ng/ml their IL-6 values are subjected to a cut-off of 15 μg/ml. If this value is greater than 15 μg/ml they are categorised as likely having NAFLD/NASH, if this value is less than 15 μg/ml their TNFα values are subjected to a cut-off of 3.7 pg/ml. If their TNFα value is less than 3.7 pg/ml then they are categorised as a control, i.e. not having benign steatosis or NAFLD/NASH. If their TNFα value is greater than 3.7 pg/ml a further IL-6 cut-off of 1.7 pg/ml is applied to determine if they are categorised as benign steatosis or NAFLD/NASH.

### Methods

### Patient Cohort and Sample Collection

One hundred and nineteen patients were included in the study. The patient cohort consisted of three groups: control (n=34), benign steatosis (n=24), and NAFLD/NASH (n=61). Patient serum samples were obtained from Discovery Life Sciences (DLS), California, US. Patient samples were de-identified and publicly available and were thus exempt from the requirement of the Institutional Review Board (IRB) approval (Exempt Category 4, IRB/EC). Samples were procured pursuant to informed consent provided by the individual under approved protocols 45 CFR 46.116. Serum (1 ml) with clinical history was obtained for each DLS patient. Samples were selected based on ICD-10 codes for liver-related conditions.

### Patient Demographics

Patient demographics are shown in Table 1.

TABLE 1
Patient Demographics

| Factor | Control | | Benign Steatosis | | NAFLD/NASH | | p value |
|---|---|---|---|---|---|---|---|
| Ethnicity (White) | 22/34 | (64.7%) | 11/13 | (84.6%) | 45/58 | (77.6%) | 0.220 |
| Gender (M) | 21/34 | (61.8%) | 6/14 | (42.9%) | 31/61 | (50.8%) | 0.419 |
| Age | 54.6 ± 15.3 | (n = 34) | 53.9 ± 10.3 | (n = 14) | 51.6 ± 14.1 | (n = 61) | 0.143 |

| | | | | | | |
|---|---|---|---|---|---|---|
| BMI | 31.9 ± 13.6 | (n = 33) | — | 34.6 ± 8.6 | (n = 40) | 0.022 |

### Biomarker Analysis

Patient samples were analysed in duplicate by Randox laboratory Clinical Services (RCLS), Antrim, UK by scientists blinded to patient data. In total, 12 biomarkers were investigated by Biochip Array Technology (BAT) (Randox Laboratories Ltd, Crumlin, UK) using the Evidence Investigator analyser, following manufacturer's instructions. The limits of detection (LOD) for the biomarkers on the biochip arrays were: EGF 2.5 pg/ml, IFNγ 2.1 pg/ml, IL-1a 0.9 pg/ml, IL-1ß 1.3 pg/ml, IL-2 4.9 pg/ml, IL-4 3.5 pg/ml, IL-6 0.4 pg/ml, IL-8 2.3 pg/ml, IL-10 1.1 pg/ml, MCP-1 25.5 pg/ml, TNFα 3.7 pg/ml, and VEGF 10.8 pg/ml. Biomarkers below the LOD were recorded as 90% of the LOD.

Three biomarkers, albumin, AST and ALT were measured on the Rx Imola (Randox Laboratories Ltd, Crumlin, UK).

Five biomarkers were analysed using commercial ELISAs, as per manufacturer's instructions: PIIINP (Cusabio, Houston, US), Midkine (LyraMid, Sydney, Australia), ST2/IL-33R (R&D Systems, Cambridge, UK), FABP-1 (Abcam, Cambridge, UK) and ApoF (LSBio, Seattle, US). The mean detectable dose (MDD) for the ELISA kits were 78 μg/ml, 8 μg/ml, 5.1 pg/ml, 1.0 ng/ml, and 0.41 ng/ml, respectively.

### Statistical Analyses

Statistical analyses were undertaken using R version 4.0.5. Wilcoxon rank sum test was used to identify differentially expressed biomarkers. Biomarkers with a p<0.05 were considered significant.

### Results

Biomarker results, BAT, Rx Imola, and ELISA, are described in Tables 2, 3 and 4, respectively.

TABLE 2
Biomarker Results (High-Sensitivity Cytokine 1 Array)

| | Control | Benign Steatosis | NAFLD/NASH | p |
|---|---|---|---|---|
| Biomarker | (n = 34) | (n = 24) | (n = 55) | value |
| EGF (pg/ml) | 59.7 ± 62.9 | 62.0 ± 41.3 | 46.7 ± 56.0 | 0.094 |
| INFγ (pg/ml) | 0.6 ± 1.0 | 1.4 ± 5.8 | 0.2 ± 0.3 | 0.021 |
| IL-1α (pg/ml) | 0.2 ± 0.2 | 0.1 ± 0.1 | 0.1 ± 0.1 | 0.271 |
| IL-1β (pg/ml) | 1.2 ± 1.3 | 0.8 ± 0.4 | 2.5 ± 7.7 | 0.029 |
| IL-2 (pg/ml) | 1.2 ± 2.0 | 0.7 ± 1.1 | 0.7 ± 1.4 | 0.079 |
| IL-4 (pg/ml) | 1.6 ± 1.3 | 1.5 ± 0.8 | 1.2 ± 0.4 | 0.062 |
| IL-6 (pg/ml) | 2.7 ± 2.8 | 5.0 ± 14.5 | 31.0 ± 72.5 | <0.001 |
| IL-8 (pg/ml) | 20.2 ± 18.8 | 35.3 ± 46.7 | 105.0 ± 162.9 | <0.001 |
| IL-10 (pg/ml) | 0.7 ± 0.7 | 0.7 ± 0.8 | 1.1 ± 1.8 | 0.037 |
| MCP-1 (pg/ml) | 163.2 ± 90.1 | 185.8 ± 118.5 | 224.1 ± 134.0 | 0.082 |
| TNF-α (pg/ml) | 3.5 ± 1.5 | 5.0 ± 2.2 | 5.6 ± 3.2 | 0.001 |
| VEGF (pg/ml) | 103.8 ± 105.4 | 96.2 ± 69.2 | 105.5 ± 107.4 | 0.916 |

TABLE 3
Biomarker Results (Rx Imola)

| | Control | Benign Steatosis | NAFLD/NASH | |
|---|---|---|---|---|
| Biomarker | (n = 34) | (n = 24) | (n = 61) | p value |
| Albumin (g/l) | 41.3 ± 4.3 | 49.3 ± 10.3 | 38.3 ± 8.5 | <0.001 |
| AST (U/I) | 24.7 ± 16.0 | 37.3 ± 26.3 | 80.2 ± 178.0 | <0.001 |
| ALT (U/I) | 23.5 ± 28.2 | 34.6 ± 35.1 | 75.9 ± 180.2 | 0.003 |

TABLE 4
ELISA Results

| | | Benign | NAFLD/ | |
|---|---|---|---|---|
| | Control | Steatosis | NASH | p |
| Biomarker | (n = 34) | (n = 24) | (n = 61) | value |
| PIIINP | 3.5 ± 5.8 | 2.9 ± 1.8 | 5.8 ± 7.9 | 0.002 |
| (ng/ml) | | | | |
| MK (pg/ml) | 720.9 ± 1130.1 | 550.7 ± 825.3 | 940.7 ± 1401.4 | 0.595 |
| ST2/IL-33R | 14.7 ± 7.4 | 14.1 ± 11.5 | 27.7 ± 19.9 | 0.001 |
| (ng/ml) | | | | |
| FABP-1 | 52.0 ± 56.6 | 33.8 ± 44.9 | 44.9 ± 42.3 | 0.129 |
| (ng/ml) | | | | |
| ApoF | 7.7 ± 9.7 | 5.9 ± 3.8 | 4.9 ± 3.7 | 0.200 |
| (μg/ml) | | | | |

### CONCLUSIONS

1. HS cytokine 1 biochip can potentially identify the progression from benign steatosis to the early stages of inflammation seen in NASH
   IL-6
   IL-8
   TNFα
2. PIIINP and ST2/IL-33R are potential markers of fibrosis
3. ST2/IL-33R looks to be a promising biomarker for grading the level of fibrosis

1. A method comprising determining the level of suppression of tumorigenicity 2 receptor (ST2) in an ex vivo blood, serum or plasma sample previously obtained from a patient suspected of having or being at risk of developing non-alcoholic steatohepatitis (NASH) or non-alcoholic fatty liver disease (NAFLD).

2. The method of claim 1, wherein the levels of one or more of the following biomarkers are also determined; interleukin 6 (IL-6), interleukin 8 (IL-8), tumour necrosis factor alpha (TNFα) and Procollagen III N-terminal peptide (P3NP) and their significance is also established in comparison to controls.

3. The method of claim 2, wherein the level of TNFα is determined.

4. The method of claim 2, wherein the levels of TNFα and IL-6 are determined.

5. The method of claim 2, wherein the levels of IL-6, IL-8, TNFα and P3NP are determined.

6. The method of claim 1, wherein each of the biomarker concentration values is inputted into a statistical methodology to produce an output value that indicates whether the patient has or is at risk of developing NASH or NAFLD.

7. The method of claim 1, used to differentiate between benign steatosis and NASH or NAFLD.

8. The method of claim 1, used to grade the fibrosis associated with NAFLD or NASH.

9. A method comprising determining the level of suppression of TNFα and ST2 in an ex vivo blood, serum or plasma sample previously obtained from a patient with benign steatosis.

10. The method of claim 9, further comprising determining the level of one or more of IL-6, IL-8 or P3NP.

11. A method comprising determining the level of TNFα, and optionally one or both of IL-6 and IL-8, in an ex vivo blood, serum or plasma sample previously obtained from a patient with benign steatosis.
